(19) United States
(12) Patent Application Publication
(10) Pub. No.: US 2014/0106977 A1
(43) Pub. Date: Apr. 17, 2014

(54) SIMULTANEOUS DETECTION OF MULTIPLE MUTATIONS

(71) Applicants: Feng GAO, Durham, NC (US); Chunfu Yang, Atlanta, GA (US); Fangping Cai, Durham, NC (US); Guoqing Zhang, Atlanta, GA (US)

(72) Inventors: Feng GAO, Durham, NC (US); Chunfu Yang, Atlanta, GA (US); Fangping Cai, Durham, NC (US); Guoqing Zhang, Atlanta, GA (US)

(21) Appl. No.: 13/909,400

(22) Filed: Jun. 4, 2013

Related U.S. Application Data
(60) Provisional application No. 61/655,216, filed on Jun. 4, 2012.

(51) Int. Cl. C12Q 1/68

(52) U.S. Cl. CPC C12Q 1/6883; C12Q 1/6858; C12Q 1/6888
USPC 506/9; 506/16

(57) ABSTRACT

Methods and compositions for simultaneous detection of polymorphisms at multiple loci in a target nucleic acid.

### FEDERAL FUNDING

The present invention was developed in part using U.S. federal government support under Federal Grant No. RO1 GM 065057 awarded by the National Institute of Health as well as funding from the U.S. President's Emergency Plan for AIDS Relief (PEPFAR) and the Association of Public Health Laboratories (APHL). Therefore, the U.S. federal government may have certain rights in the invention.

### FIELD OF THE INVENTION

The present invention generally relates to methods and compositions for detecting multiple polymorphisms in a target nucleic acid.

### BACKGROUND

Single nucleotide polymorphisms (SNPs) are a significant form of genetic variation, and functional SNPs encompass the majority of mutant alleles that cause or predispose to human disease and drug resistance. Thus, there is a continuing effort to discover SNPs as well as to develop methods for their rapid detection. Technology for simultaneous detection of multiple mutations would be particularly beneficial in developing treatments for disorders or conditions with significant allelic heterogeneity, and for treatment of infectious diseases in which many drug resistant mutations are known. As one example, surveillance of HIV drug resistant mutations (HIVDR) will enable countries and regions to track trends in HIV drug resistance, make rational choices in containment activities, and provide effective treatments. More than 20 drugs have been approved for treatment of HIV infection, which target a number of viral genes. When used in combinations, they have greatly improved patients' health quality and life span. Nonetheless, some patients still fail antiretroviral therapy due to drug resistance. A number of common genotypic assays are presently used to detect drug resistant mutations, but the existing assays are sophisticated, labor intensive, time consuming, and/or costly. In addition, assays such as real-time PCR, hybridization, and ligation PCR, can only detect one or very few mutations at a time. Thus, a rapid, high throughput, accurate and inexpensive genotyping method is urgently needed. To this end, the present invention provides a multiplexing allele-specific HIVDR mutation assay (MASHMA) based upon suspension array technology.

### SUMMARY

The present invention provides a method of simultaneously genotyping polymorphic bases at two or more loci of a target nucleic acid. In one aspect of the invention, the method comprises the steps of (a) obtaining a target nucleic acid; (b) preparing a reaction mixture comprising the target nucleic acid and a plurality of allele specific primer extension primers that specifically hybridize to the two or more loci of the target nucleic acid, in conditions sufficient for hybridization, primer extension, and labeling with a reporter molecule; wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence; (c) annealing the primer extension products of (b) to a plurality of beads, wherein each bead comprises (i) a second unique allele specific sequence that specifically hybridizes to the first unique allele specific sequence, and (ii) a unique detectable label; (d) detecting the reporter molecule; and (e) detecting the unique detectable label.

Also provided are compositions comprising a plurality of allele specific primer extension primers that specifically hybridize to two or more loci of a target nucleic acid, wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence. Such kits can further comprise one or more of reagents for extraction of nucleic acids comprising the target nucleic acid, PCR primers for amplification of the target nucleic, deoxynucleotides, enzymes, and buffers.

Still further provided are kits for simultaneously genotyping polymorphisms at multiple loci in a target nucleic acid. A representative kit comprises (a) a plurality of allele specific primer extension primers that specifically hybridize to two or more loci of a target nucleic acid, wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence; and (b) a plurality of beads, wherein each bead comprises (i) a second unique allele specific sequence that specifically hybridizes to the first unique allele specific sequence, and (ii) a unique detectable label.

In some aspects of the invention, target nucleic acids used in the disclosed genotyping methods are associated with drug resistance, such as antiretroviral drug resistance, or resistance to a drug for the treatment of HIV infection, hepatitis B infection, hepatitis C infection, tuberculosis, or other infection. For example, a target nucleic acid may be associated with resistance to one or more of a nucleoside reverse transcriptase inhibitor, a non-nucleoside reverse transcriptase inhibitor, and a protease inhibitor. In other aspects of the invention, the target nucleic acid is a pathogen nucleic acid, such as a viral nucleic acid, or more particularly, an HIV nucleic acid. For example, a representative target nucleic acid is an HIV-1 pol gene, or fragment thereof, such as fragments encoding a reverse transcriptase or a protease. Target nucleic acids used in the disclosed genotyping methods are obtainable from human subjects, including subjects who have received antiretroviral therapy, treatment naïve subjects, subjects who are candidates for antiretroviral therapy, HIV-infected subjects, healthy subjects, or any other subpopulation.

In one aspect of the invention, the plurality of allele specific primer extension primers used for performing the disclosed genotyping assay, or for preparing the disclosed compositions and kits, comprises two or more primers that specifically hybridize to polymorphic bases of a pathogen target nucleic acid, which polymorphic bases are at two or more loci corresponding to drug resistant mutations of the target nucleic acid. For example, the plurality of allele specific primer extension primers can comprise primers that specifically hybridize to a polymorphic base selected from any one of V32I, M41L, I47A/V, K65R, D67N, K70R, L74V, L76V, I84V, L90M, L100I, K101P, K103N, V106A/M, Y115F, Q151M, Y181C, M184V, Y188L, G190A, L210W, T215F/Y, and K219Q/E of an HIV-1 pol gene. As another example, the plurality of allele specific primer extension primers can comprise primers that specifically hybridize to a polymorphic base selected from two or more of V32I, M41L, I47A/V, K65R, D67N, K70R, L74V, L76V, I84V, L90M, L100I, K101P, K103N, V106A/M, Y115F, Q151M, Y181C, M184V, Y188L, G190A, L210W, T215F/Y, and K219Q/E of an HIV-1 pol gene. As another example, the plurality of allele specific primer extension primers can comprise primers that specifically hybridize to polymorphic bases comprising V32I, M41L, I47A/V, K65R, K70R, L74V, L76V, I84V, L90M, L100I, K101P, K103N, V106A/M, Y115F, Q151M, Y181C, M184V, Y188L, G190A, and K219Q/E of an HIV-1 pol gene. As another example, the plurality of allele specific primer extension primers can comprise at least a first primer and a second primer, wherein each primer comprises a nucleotide sequence of any one of SEQ ID NOs: 1-45. As another example, the plurality of allele specific extension primers can comprise primers set forth as SEQ ID NOs: 1-45. As another example, the plurality of allele specific primer extension primers that specifically hybridize to the two or more loci can comprise at least one allele specific primer extension primer set, wherein a primer set comprises at least a first primer that hybridizes to a wild type allele comprising a first polymorphic base at a locus and a second primer that hybridizes to a mutant allele comprising a second polymorhphic base at the same locus. The primer set can further comprise a third primer that hybridizes to a mutant allele comprising a third polymorphic base at the same locus.

Further with respect to the allele specific primers used in the genotyping methods and compositions of the invention, the first unique allele specific sequence (found at or near the 5′ end of each allele specific primer extension primer) comprises at least about 18 nucleotides.

The disclosed genotyping method is performed using well known conditions for hybridization, primer extension, and labeling with a reporter molecule. In one aspect of the invention, conditions sufficient for labeling with a reporter molecule can comprise addition of a reporter molecule such as a biotinylated deoxynucleotide to the reaction mixture, which is thereafter detected using a streptavidin-linked selection medium.

For performing the multiplex analysis step of the disclosed method, a plurality of beads is used, such as microspheres. Each bead comprises a unique detectable label, such as a label that is detectable using laser-based fluorescent analysis.

### BRIEF DESCRIPTION OF THE DRAWINGS

FIG. 1 is a series of bar graphs showing detection of both wild type and drug resistant mutations by MASHMA. After PCR amplification of each template individually, wild type template, mutant template, a 1:1 mixture of the wild type and mutant templates, and a blank control were analyzed with 45 ASPE primers in one reaction. The results from five independent assays are shown. See Example 1. MFI, median fluorescence intensity; WT, wild type template; mutant, mutant template; mix, a 1:1 mixture of the wild type and mutant templates; NC, blank control.

FIG. 2 is a series of bar graphs showing the sensitivity of detection of minority drug resistant mutations by MASHMA. Assays were performed using serial dilutions (1:2 ratio) of mutant template as indicated. The results of three independent assays are shown. See Example 1. MFI, median fluorescence intensity; NC, background; neg, blank control.

### DETAILED DESCRIPTION

The present invention provides methods and compositions for simultaneous detection of polymorphisms at multiple loci in a target nucleic acid. As compared to existing genotyping assays, the methods disclosed herein employ a single target nucleic acid template and reaction mixture for multiplexed assaying of multiple polymorphisms in the target nucleic acid. These design differences enable numerous benefits, including rapid data acquisition, excellent sensitivity and specificity, limited sample requirements, ease of use, and reduction of cost.

A practical application of the disclosed genotyping methods is for detecting and monitoring drug resistant mutations, particularly in the case of disease caused by rapidly evolving pathogens. Existing drugs for the treatment of HIV infection target four viral genes: protease, reverse transcriptase, integrase, and envelope. When used in combinations, these drugs have greatly improved patients' health quality and life span. Nonetheless, some patients still fail antiretroviral therapy (ART) due to drug resistance. The emergence of HIV drug resistance in some treated patients is inevitable even if appropriate ART regimens are provided and optimal adherence to therapy is supported due to the error prone reverse transcriptase, fast turnover of viral populations, and the need for lifelong treatment.

In general, there are two types of drug resistant mutations: acquired and transmitted. Acquired drug resistant mutations are selected under drug pressure in individuals receiving ART. They can result from poor adherence, treatment interruptions, inadequate plasma drug concentrations, or the use of suboptimal drug combinations. Transmitted drug resistant mutations occur when individuals are infected with viruses from patients who have developed drug resistance. Studies show that drug resistant mutations are present in over one-third of the treated patients and one-tenth of treatment naïve individuals. Thus, it is critical to detect drug resistant mutations in both populations for population-based surveillance, monitoring emergency of drug resistant mutations in treated patients, and baseline tests to optimize treatment regimens by avoiding use of drugs to which patients have developed resistance.

There are less than 50 primary well-defined drug resistant mutations to current antiretroviral drugs. By taking advantage of the multiplex feature of suspension array technology, the present invention provides an assay for detecting all known antiretroviral drug resistant mutations in 1 reaction per sample, although it is possible that 2 or more reactions may be required when additional mutations are analyzed.

In one aspect of the invention, a method of simultaneously genotyping multiple mutations comprises the steps of (a) obtaining a target nucleic acid; (b) preparing a reaction mixture comprising the target nucleic acid and a plurality of allele specific primer extension primers that specifically hybridize to two or more loci of the target nucleic acid, in conditions sufficient for hybridization, primer extension, and labeling with a reporter molecule, wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence; (c) annealing the primer extension products of (b) to a plurality of beads, wherein each bead comprises (i) a second unique allele specific sequence that specifically hybridizes to the first unique allele specific sequence, and (ii) a unique detectable label; (d) detecting the reporter molecule; and (e) detecting the unique detectable label.

Target nucleic acids that can be used in the disclosed assay include any nucleic acid containing polymorphisms. Nucleic acids are deoxyribonucleotides or ribonucleotides and polymers thereof in single-stranded, double-stranded, or triplexed form, such as genes, cDNAs, mRNAs, and cRNAs. Target nucleic acids contain multiple single nucleotide polymorphisms (SNPs), i.e., sequence variations occurring when a single nucleotide—A, T, C or G—in the genome (or other shared sequence) differs between members of a biological species or between paired chromosomes in an individual. Polymorphisms can be naturally occurring variations or mutations.

In accordance with the disclosed methods, a target nucleic acid is obtained for performing the assay. Such target nucleic acids may be synthesized, or may be derived from any biological source, including any organism. In some aspects of the invention, a target nucleic acid is derived from a clinical sample, such as blood, urine, or cell and tissue samples (e.g., obtained by biopsy, aspiration, swabbing, spinal tap). In the context of clinical samples, subjects providing such samples may be healthy, treatment naïve, or undergoing particular therapies (e.g., ART), or have previously received such therapies. For example, a subject may be infected with a pathogen, or is suspected of being infected with a pathogen. In other aspects of the invention, the sample is an environmental sample such as a water, soil, or air sample. In still other aspects of the invention, the sample is from a plant, bacteria, virus, fungi, protozoan, or metazoan. For use in the disclosed primer extension reaction, a target nucleic acid may be isolated, purified, amplified, extracted, or otherwise procured from a biological sample for use in the assay using well known techniques. See e.g., Sambrook et al. (eds.) (1989) Molecular Cloning: A Laboratory Manual, Cold Spring Harbor Laboratory Press, Cold Spring Harbor, N.Y.; Silhavy et al. (1984) Experiments with Gene Fusions, Cold Spring Harbor Laboratory Press, Cold Spring Harbor, N.Y.; Glover & Hames (1995) DNA Cloning: A Practical Approach, 2nd ed., IRL Press at Oxford University Press, Oxford/New York; Ausubel (ed.) (1995) Short Protocols in Molecular Biology, 3rd ed., Wiley, New York. Prior to performing a primer extension reaction in accordance with the disclosed methods, a target nucleic acid may be stored, frozen, transferred, or otherwise handled in a manner that generally preserves the integrity of the nucleic acid.

The target nucleic acid may be any sequence of interest containing multiple SNPs. In general, such sequences will be known to have a relatively high density of SNPs and/or rapidly evolving sequences. In some aspects of the invention, target nucleic acids contain SNPs that are associated with human health conditions, such as genetic disorders having pronounced allelic heterogeneity. For example, at least 272 cystic fibrosis mutations are known, of which 55 are common. See e.g., Estivill et al., Hum. Mutat., 10: 135-154.

Target nucleic acids containing SNPs associated with human health conditions also include non-human nucleic acids present in human subjects, for example, nucleic acids from viruses, bacteria, fungi, and other pathogens. In particular aspects of the invention, a target nucleic acid is associated with resistance to a drug for the treatment of HIV, hepatitis B infection, hepatitis C infection, tuberculosis, or other infection. In the context of HIV infection, relevant target nucleic acids include those of HIV-1 and HIV-2, including groups, subtypes, subsubtypes, and circulating recombinant forms thereof, as are known in the art. As one example, a target nucleic acid useful in the disclosed methods is an HIV-1 pol gene (SEQ ID NO: 46), or fragment thereof, such as fragments encoding reverse transcriptase, integrase, and/or protease enzymes.

As described herein, a beneficial feature of the disclosed assay design is simultaneous detection of a plurality of polymorphisms at two or more loci in a single target nucleic acid. Notwithstanding the foregoing, it is contemplated that the disclosed genotyping assay may be modified to include a two or more target nucleic acids, while generally still being a relatively small number of target nucleic acids, and wherein one of the target nucleic acids comprises multiple polymorphisms at different loci that are simultaneously detected in the assay. As one example, a genotyping assay may include HIV-1 pol and HIV-1 env target nucleic acids in a single reaction mixture. As another example, a genotyping assay may include HIV-1 pol, env, and gag target nucleic acids in a single reaction mixture.

In accordance with the disclosed methods, at least two different primers are used in a single reaction mixture to detect at least two different polymorphic bases at two different loci in a target nucleic acid. As used herein, the phrase “different loci” refers to any residues of a target nucleic acid other than complementary bases found at a same position in the target nucleic acid. A residue “position” within a target nucleic acid refers to linear placement of the residue, taking into account the context of the entire target nucleic acid sequence. Thus residues at a same position are complementary and form a hydrogen bond with each other in the context of a double-stranded target nucleic acid.

Complementarity is a property of double-stranded nucleic acids such as DNA, as well as DNA:RNA duplexes. Each strand is complementary to the other in that the base pairs between them are non-covalently connected via two or three hydrogen bonds. For DNA, adenine (A) bases complement thymine (T) bases and vice versa; guanine (G) bases complement cytosine (C) bases and vice versa. With RNA, it is the same except that adenine (A) bases complement uracil (U) bases instead of thymine (T) bases.

For example, in the sequence shown below, residues marked with a plus sign are found at different loci, and residues marked with an asterisk are found at a same locus.

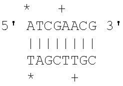

The disclosed methods may be used to detect polymorphic bases at two or more different loci, and in the same reaction mixture, also detect polymorphic base(s) at the same loci, i.e., all of the bases marked with an asterisk and all of the bases marked with a plus sign in the example above.

Thus, a single reaction mixture will include at least two ASPE primers that anneal to a same target gene, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more primers.

Primers of the invention have the above-enumerated properties, namely, each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence. Unique allele specific sequences are used as a component of multiplex analysis, as described herein below.

The ASPE primers may otherwise incorporate well-known features of primer design, such as minimal secondary structure (e.g., hairpins and other interstrand structure) and sufficient complexity for sequence identification. In general, primers used in the disclosed methods will be about 18 to about 70 nucleotides in length. Unless specifically limited, primers may contain known analogues of natural nucleotides that have similar properties as the reference natural nucleic acid. See e.g., Robertson et al., Methods Mol. Biol., 1998, 98: 121-54; Rychlik et al., Nucleic Acids Research, 1989, 17: 8543-8551 (1989); and Breslauer et al., Proc. Natl. Acad. Sci. USA, 1986, 83: 3746-3750.

ASPE primers used in the disclosed genotyping assay may be degenerate to optimize annealing of primers to a target nucleic acid having closely positioned polymorphic bases, i.e., polymorphic bases at positions within the hybrid (i.e., the hybridized region of ASPE primer and target nucleic acid) other than the primer 3′ terminus. See e.g., Example 1 and Table 3.

ASPE primers used together in a single reaction mixture should have similar annealing temperatures (Ta) and sufficiently different sequences so that they to not hybridize to one another to form heterodimers. The optimal annealing temperature (Ta) is the range of temperatures where efficiency of primer annealing and extension is maximal without non-specific products. Relevant values for estimating the Ta include primer quality and melting temperatures (Tm) of the primers. Primers with a relatively high Tm (e.g., >60° C.) can be used in reactions with a wide Ta range as compared to primers with relatively low Tm e.g., >50° C.) Thus, the optimal annealing temperature for the primer extension reaction is calculated directly as the value for the primer with the lowest Tm (Tm). For hybrids less than 18 base pairs in length, Tm (° C.) is calculated as the product of 2(# of A+T bases)+4(# of C+G bases). For hybrids between 18 and 49 base pairs in length, Tm (° C.) is calculated as the product of 81.5+16.6(log₁₀ Na⁺)+0.41(percentage of G+C bases in the hybrid)−(600/# of bases in the hybrid), wherein Na⁺ is the concentration of sodium ions in the hybridization buffer. The primer extension reaction may be performed at temperatures up to 10 degrees (° C.) higher than the Tm of the primer to favor primer/target duplex formation.

Software tools may be employed for primer design, including design of primers for use in a single reaction, such as the PRIMERPLEX® (Premier Biosoft) and FASTPCR® (PrimerDigital) software.

A representative plurality of primers that can be used together in a single reaction include the primers set forth as SEQ ID NOs: 1-45. See Examples 1 and 2. In some aspects of the invention, a subset of SEQ ID NOs: 1-45 are used in a single reaction, or SEQ ID NOs: 1-45 may be used in combination with additional primers that are compatible, i.e., show specific hybridization when used in a single reaction. In other aspects of the invention, one or more bases of any one of SEQ ID NOs: 1-45 is changed while maintaining specific hybridization to a target nucleic acid.

In preparing a reaction mixture comprising the target nucleic acid and a plurality of ASPE primers that specifically hybridize to the two or more loci of the target nucleic acid, well-known conditions sufficient for annealing, primer extension, and labeling with a reporter molecule are used. “hybridizing” or “annealing” refers to the binding, annealing, duplexing, or hybridizing of a probe (e.g., ASPE primer or allele specific sequence) only to a particular nucleotide sequence under stringent or highly stringent conditions when that sequence is present in a complex nucleic acid mixture. Specific hybridization may accommodate mismatches between the probe and the target sequence depending on the stringency of the hybridization conditions. In the allele specific primer extension reaction, polymerases extend the primer by incorporating dNTPs. Extension only occurs if the 3′ end of the ASPE primer is bound to the homologous allelic sequence. One skilled in the art can readily determine an appropriate temperature based upon the optimal annealing temperature (Ta) of the primers used in the reaction. See Table 1 below. For example, the Ta of the primers may be within the range from about 45° C. to about 68° C.

TABLE 1
Hybridization/Annealing Conditions

| | Poly- | Hybrid | Hybridization | Wash |
|---|---|---|---|---|
| Stringency | nucleotide | Length | Temperature and | Temperature |
| Condition | Hybrid | (base pairs) | Buffer | and Buffer |
| Highly | DNA:DNA | ≧50 | 65° C.; 1X SSC | 65° C.; |
| stringent | | | -or- | 0.3X SSC |
| | | | 42° C.; 1X SSC, | |
| | | | 50% formamide | |
| Highly | DNA:DNA | <50 | Tm; 1X SSC | Tm; 1X SSC |
| stringent | | | | |
| Highly | DNA:RNA | ≧50 | 67° C.; 1X SSC | 67° C.; |
| stringent | | | -or- | 0.3X SSC |
| | | | 45° C.; 1X SSC, | |
| | | | 50% formamide | |
| Highly | DNA:RNA | <50 | Tm; 1X SSC | Tm; 1X SSC |
| stringent | | | | |
| Highly | RNA:RNA | ≧50 | 70° C.; 1X SSC | 70° C.; |
| stringent | | | -or- | 0.3X SSC |
| | | | 50° C.; 1X SSC, | |
| | | | 50% formamide | |
| Highly | RNA:RNA | <50 | Tm; 1X SSC | Tm; 1X SSC |
| stringent | | | | |
| Stringent | DNA:DNA | ≧50 | 65° C.; 4X SSC | 65° C.; |
| | | | -or- | 1X SSC |
| | | | 42° C.; 4X SSC, | |
| | | | 50% formamide | |
| Stringent | DNA:DNA | <50 | Tm; 4X SSC | Tm; 4X SSC |
| Stringent | DNA:RNA | ≧50 | 67° C.; 4X SSC | 67° C.; |
| | | | -or- | 1X SSC |
| | | | 45° C.; 4X SSC, | |
| | | | 50% formamide | |
| Stringent | DNA:RNA | <50 | Tm; 4X SSC | Tm; 4X SSC |
| Stringent | RNA:RNA | ≧50 | 70° C.; 4X SSC | 70° C.; |
| | | | -or- | 1X SSC |
| | | | 50° C.; 4X SSC, | |
| | | | 50% formamide | |
| Stringent | RNA:RNA | <50 | Tm; 2X SSC | Tm; 2X SSC |

The reaction mixture also includes a reverse transcriptase enzyme, such as AMV RT (avian myeloblastosis virus reverse transcriptase), MMLV RT (Moloney murine leukemia virus reverse transcriptase), SUPERSCRIPT® II (BRL), and RETROTHERM® (Epicentre Technologies). The efficiency of the primer extension reaction may be altered or optimized by varying the reaction mixture components (e.g., substitution of known buffers, addition of trehalose or other components) and/or by adjusting the reaction temperature, as is well known in the art. See e.g., Carninci et al., Proc. Natl. Acad. Sci. USA, 1998, 95: 520-524; Mizuno et al., Nucleic Acids Research, 1999, 27: 1345-1349; and Pastinen et al., Genome Research, 200, 10: 1031-1042.

Conditions sufficient for labeling of the ASPE reaction products are conditions whereby a reporter molecule selectively labels products of the ASPE reaction. For example, dNTPs bearing a reporter molecule may be included in the ASPE reaction mixture. The target nucleic acid specifically lacks the reporter molecule. The labeling may be direct in that the reporter molecule is immediately detectable (e.g., fluorescent dNTPs). Alternatively, the labeling may be indirect in that binding to a second reporter molecule, contacting with an enzyme substrate, etc. is performed for detecting of the reporter molecule. In one aspect of the invention, a biotinylated dNTP is included in the reaction mixture for labeling of the ASPE reaction product, which is later detected by binding to a streptavidin-linked selection medium.

In accordance with the invention, the products of the primer extension reaction are used without further purification in a multiplex analysis system. Specifically, the primer extension reaction mixture is contacted with a plurality of beads, wherein each bead comprises (i) a second unique allele specific sequence that specifically hybridizes to the first unique allele specific sequence (which is at or near the 5′ end of each primer) and (ii) a unique detectable label.

A “bead” as used herein is any solid phase particle that may be used in suspension. In general, a bead is substantially spherical and may vary in size to include microspheres (˜5-40 μm in diameter), nanoparticles (particles having one or more dimensions on the order of 100 nm or less), etc. The beads may be composed of any appropriate material, including for example, plastic, glass, silicon, nylon, polystyrene, silica gel, latex and the like.

Each bead comprises a unique allele specific sequence that specifically hybridizes to a unique allele specific sequence included at or near the 5′ end of each ASPE primer. Specifically hybridizing allele specific sequences include sequences that are substantially identical or have a relatively high percentage of sequence identity. In some aspects of the invention, specifically hybridizing allele specific sequences are reverse complementary, i.e., a complementary, antisense strand sequence (3′ to 5′) rewritten in the direction 5′ to 3′. Specifically hybridizing allele specific sequences hybridize to one another under stringent or highly stringent conditions. See Table 1.

The unique allele specific sequence pairs (a first sequence at or near the 5′ end of an ASPE primer and a second sequence, which is reverse complementary to the first, on a bead) are used to simultaneously detect ASPE reaction products, which have been labeled with a reporter molecule, and which uniquely identify each of the plurality of polymorphisms in the target nucleic acid. Given the multiplex nature of the assay, allele specific sequences used in a single reaction must be unique in that they show minimal cross-hybridization with one another under stringent or highly stringent hybridization conditions (see Table 1). Allele specific sequences may be degenerate so long as the degenerate bases do not permit cross-hybridization among allele specific sequences used in a same reaction.

Allele specific sequences are generally at least about 18-20 nucleotides in length, for example, at least about 25 nucleotides, such as about 25 nucleotides, about 30 nucleotides, about 35 nucleotides, about 40 nucleotides, about 45 nucleotides, about 50 nucleotides, about 55 nucleotides, about 60 nucleotides, about 65 nucleotides, or about 70 nucleotides. Allele specific sequences used in a same reaction may have different lengths, but should have similar annealing temperatures (see discussion of Ta and Tm with respect to ASPE primers and Table 1 above).

Allele specific sequences that do not substantially hybridize with other such sequences in a same reaction can be empirically determined or can be derived using a computer program, for example, as described in PCT International Publication No. WO 01/59151. A rational design approach for determining allele specific sequences may be performed as follows. A set of sequences of a given length are created based on a given number of block elements. If a family of polynucleotide sequences 24 nucleotides (24 mer) in length is desired from a set of 6 block elements, each element comprising 4 nucleotides, then a family of 24 mers is generated considering all positions of the 6 block elements. In this case, there will be 6⁶ (46,656) ways of assembling the 6 block elements to generate all possible polynucleotide sequences 24 nucleotides in length. Constraints are imposed on the sequences and are expressed as a set of rules on the identities of the blocks such that homology between any two sequences will not exceed the degree of homology desired between these two sequences, generally about 35-70%. All polynucleotide sequences generated which obey the rules are saved. Sequence comparisons are performed in order to generate an incidence matrix. The incidence matrix is presented as a simple graph, and the sequences with the desired property of being minimally cross hybridizing are found from a clique of the simple graph, which may have multiple cliques. Once a clique containing a suitably large number of sequences is found, the sequences are experimentally tested to determine if it is a set of minimally cross hybridizing sequences. Block sequences can be composed of all natural bases, a subset of natural bases, or a combination of natural and synthetic bases.

In some aspects of the invention, a plurality of beads, wherein each bead comprises a unique allele specific sequence, may be purchased from a vendor. For example, xTAG® sequences (Luminex Corporation) may be used as allele specific sequences in accordance with the disclosed invention. See Examples 1 and 2.

Allele specific sequences may be bound to the bead through covalent or non-covalent bonds. See e.g., Iannone et al., Cytometry, 2000, 39: 131-140; Matson et al., Anal. Biochem., 1995, 224: 110-106; Proudnikov et al., Anal. Biochem., 1998, 259: 34-41; Zanimatteo et al., Anal. Biochem., 2000, 280:143-150. A variety of moieties useful for binding to a solid support (e.g., biotin, antibodies, and the like), and methods for attaching them to nucleic acids, are known in the art. For example, an amine-modified nucleic acid base may be attached to a solid support such as COVALINK-NH®, a polystyrene surface grafted with secondary amino groups using a bifunctional crosslinker (e.g., bis(sulfosuccinimidyl-suberate). Additional spacing moieties can be added to reduce steric hindrance between the allele specific sequence and the bead surface. Alternatively, the allele specific sequence may be synthesized directly on the bead.

The plurality of beads used in the assay further comprises detectable labels, wherein there is a known association between the unique allele specific sequence and unique detectable label for each bead. A unique detectable label is any labeling agent that facilitates the detection of the bead to which it is associated. Representative detectable labels include fluorophores, chromophores, and radiophores. Non-limiting examples of fluorophores include, a red fluorescent squarine dye such as 2,4-Bis[1,3,3-trimethyl-2-indolinylidenemethyl]cyclobutenediylium-1,3-dio-xolate, an infrared dye such as 2,4Bis[3,3-dimethyl-2-(1H-benz[e]indolinylidenemethyl)]cyclobutenediylium- -1,3-dioxolate, or an orange fluorescent squarine dye such as 2,4-Bis[3,5-dimethyl-2-pyrrolyl]cyclobutenediylium-1,3-dioxolate. Additional non-limiting examples of fluorophores include quantum dots, ALEXA FLUOR® dyes, AMCA, BODIPY® 630/650, BODIPY® 650/665, BODIPY®-FL, BODIPY®-R6G, BODIPY®-TMR, BODIPY®-TRX, CASCADE BLUE®, CyDyes (e.g., CY2®, Cy3®, and Cy5®) a DNA intercalating dye, 6-FAM®, Fluorescein, HEX®, 6-JOE, OREGON GREEN® 488, OREGON GREEN® 500, OREGON GREEN® 514, PACIFIC BLUE®, REG, phycobilliproteins (e.g., phycoerythrin and allophycocyanin,) RHODAMINE GREEN®, RHODAMINE RED®, ROX®, TAMRA®, TET®, Tetramethylrhodamine, and TEXAS RED®. When a same detectable label is used for a plurality of beads, a unique amount of the label may be used to distinguish each bead. A signal amplification reagent, such as tyramide (PerkinElmer), may be used to enhance the fluorescence signal. Pairs of detectable labels, such as fluorescence resonance energy transfer pairs or dye-quencher pairs, may also be employed.

The present invention also provides compositions comprising a plurality of ASPE primers useful in the genotyping assay. In a particular aspect of the invention, ASPE primers for the detection of polymorphisms within HIV-1 pol are provided, i.e., primers set forth as any one or more or all of SEQ ID NOs: 1-45. Compositions of the invention may also comprise primers set forth as any one or more or all of SEQ ID NOs: 1-45, each primer further including a unique allele specific sequence at or near its 3′ end. For example, a composition of the invention can comprise two or more ASPE primers selected from any one of SEQ ID NOs: 1-45, which bind to two or more loci, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 primers selected from SEQ ID NOs: 1-45.

Still further provided are kits for simultaneously genotyping polymorphisms at multiple loci in a target nucleic acid as described herein. A representative kit comprises (a) a plurality of allele specific primer extension primers that specifically hybridize to two or more loci of a target nucleic acid, wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence; and (b) a plurality of beads, wherein each bead comprises (i) a second unique allele specific sequence that is reverse complementary to the first unique allele specific sequence, and (ii) a unique detectable label. Such kits may also include buffers, enzymes, nucleotides, reporter molecules, and/or other reagents in appropriate amounts and volumes for performance of the assay. Such kits also include instructions for proper use of kit reagents.

### EXAMPLES

The following examples are included to illustrate modes of the invention. Certain aspects of the following examples are described in terms of techniques and procedures found or contemplated by the present co-inventors to work well in the practice of the invention. The examples illustrate standard laboratory practices of the co-inventors. In light of the present disclosure and the general level of skill in the art, those of skill will appreciate that the following examples are intended to be exemplary only and that numerous changes, modifications, and alterations may be employed without departing from the scope of the invention.

### Example 1

### Assay Development for Rapid Detection of HIV-1 Drug Resistant Mutations

A DNA fragment containing the target mutation sites were amplified by RT-nested PCR essentially as described in Zhou et al., PLoS One, 2011, 6:e28184. For each allele site, two or three allele specific primer extension (ASPE) primers were designed. Each primer was capable of hybridizing to one of the expected alleles (wild type or mutant), with the 3′ terminal nucleotide being complementary to the polymorphic base. The 5′ end of each primer carried a unique 24-base xTAG® (Luminex Corporation) sequence reverse-complementary to anti-xTAG® sequences uniquely assigned to different sets of microspheres. All of the primers for targeted polymorphic sites were mixed together for multiplex ASPE to detect both wild type and mutant bases at each site. Primers with a complementary 3′ terminal nucleotide were extended in the presence of biotinylated dCTPs. The ASPE reaction mixture was then annealed with microspheres bearing superficial anti-xTAG® nucleic acids. A LUMINEX® (Luminex Corporation) detection system was used to identify each microsphere by its unique internal dye, and the associated reporter dye intensity was recorded as mean fluorescence intensity (MFI). The MFI value uniquely identified each allele.

In one assay, ASPE primers are designed for two or more of 23 allele specific mutations associated with HIV drug resistance to nucleoside reverse transcriptase inhibitor (NRTI), non-nucleoside reverse transcriptase inhibitor (NNRTI), and protease inhibitor (PI). See Table 2.

TABLE 2
HIV-1 Drug Resistant Mutations

| Drug | n | Mutations |
|---|---|---|
| NRTIs | 11 | M41L, K65R, D67N, K70R, L74V, Y115F, |
| | | Q151M, M184V, L210W, T215F/Y, K219Q/E |
| NNRTIs | 7 | L100I, K101P, K103N, V106A/M, Y181C, |
| | | Y188L, G190A |
| PIs | 5 | V32I, I47A/V, L76V, I84V, L90M |

In another assay, forty-five (45) ASPE primers were designed for 20 allele specific mutations associated with HIV drug resistance. One primer was designed for each wild type and mutant allele at twenty (20) SNPs loci. At five (5) loci, two mutant bases are known, and therefore, an ASPE primer was designed for each mutant allele. The primer sequences and positions are shown in Table 3 below.

TABLE 3
ASPE Primer Sequences for MASHMA

| SEQ | | | | |
|---|---|---|---|---|
| ID NO. | Allele | Sequences, 5′-3′ | Tag ID | Location |
| 1 | M41 | Tag- AAG ARA AAA TAA AAG CAT TAA YAG MAA TTT GTG AWG ARA | 45 | 2632 → 2670 |
| 2 | 41L | Tag- AAG ARA AAA TAA AAG CAT TAA YAG MAA TTT GTG AWG ARC | 38 | 2632 → 2670 |
| 3 | K65 | Tag- AAA TCC ATA TAA CAC TCC ART ATT TGC YAT AAA RAA | 12 | 2708 → 2743 |
| 4 | 65R | Tag- AAA TCC ATA TAA CAC TCC ART ATT TGC YAT AAA RAG | 13 | 2708 → 2743 |
| 5 | K70 | Tag- CCA GTA TTT GCC ATA AAG ARG AAR GAY AGT ACT AA | 18 | 2724 → 2758 |
| 6 | 70R | Tag- CCA GTA TTT GCC ATA AAG ARG AAR GAY AGT ACT AG | 21 | 2724 → 2758 |
| 7 | L74 | Tag- CAT AAA AAA GAA RGA CAG TAC HAR RTG GAG AAA AT | 67 | 2735 → 2769 |
| 8 | 74V | Tag- CAT AAA AAA GAA RGA CAG TAC HAR RTG GAG AAA AG | 90 | 2735 → 2769 |
| 9 | Y115 | Tag- AGT RCT RGA YGT GGG RGA TGC ATA | 73 | 2870 → 2893 |
| 10 | 115F | Tag- AGT RCT RGA YGT GGG RGA TGC ATT | 62 | 2870 → 2893 |
| 11 | Q151 | Tag- GGA TTA GRT ATC AAT ATA ATG TRY TNC CAC | 29 | 2971 → 3000 |
| 12 | 151M | Tag- GGA TTA GRT ATC AAT ATA ATG TRY TNC CAA | 36 | 2971 → 3000 |
| 13 | M184 | Tag- AGR GCA AAA AAT CCA GAM RTR GTY ATC TRY CAA TAY A | 37 | 3063 → 3099 |
| 14 | 184V | Tag- AGR GCA AAA AAT CCA GAM RTR GTY ATC TRY CAA TAY G | 22 | 3063 → 3099 |
| 15 | K219 | Tag- AAR TGG GGR TTT ACY ACA CCA GAC A | 53 | 3180 → 3204 |
| 16 | 219Q | Tag- AAR TGG GGR TTT ACY ACA CCA GAC C | 44 | 3180 → 3204 |
| 17 | 219E | Tag- AAR TGG GGR TTT ACY ACA CCA GAK G | 96 | 3180 → 3204 |
| 18 | L100 | Tag- CAA TTA GGR ATA CCA CAC CCA KCA GGR T | 42 | 2820 → 2847 |
| 19 | 100I | Tag- CAA TTA GGR ATA CCA CAC CCA KCA GGR A | 58 | 2820 → 2847 |
| 20 | K101 | Tag- AAT TAG GRA TAC CAC ACC CAK CAG GRW TRA | 55 | 2821 → 2850 |
| 21 | 101P | Tag- AAT TAG GRA TAC CAC ACC CAK CAG GRW TRC | 89 | 2821 → 2850 |
| 22 | 101E | Tag- AAT TAG GRA TAC CAC ACC CAK CAG GRW TRG | 65 | 2821 → 2850 |
| 23 | K103 | Tag- GAA TAC CAC ACC CAK CAG GGT TRA ARA AGA AA | 19 | 2827 → 2858 |
| 24 | 103N | Tag- GAA TAC CAC ACC CAK CAG GGT TRA ARA AGA AY | 66 | 2827 → 2858 |
| 25 | 103R | Tag- GAA TAC CAC ACC CAK CAG GGT TRA ARA AGA GA | 63 | 2827 → 2858 |
| 26 | V106 | Tag- ACA CCC AKC AGG GTT AAA RAA GAA HAA RTC WGT | 20 | 2834 → 2866 |
| 27 | 106A | Tag- ACA CCC AKC AGG GTT AAA RAA GAA HAA RTC WGC | 39 | 2834 → 2866 |
| 28 | 106M | Tag- CAC ACC CAK CAG GGT TAA ARA AGA AHA ART CWA | 75 | 2833 → 2865 |
| 29 | Y181 | Tag- GCC CTT TAG RRC AMA AAA TCC AGA MVT RGT YAT CTA | 9 | 3056 → 3091 |
| 30 | 181C | Tag- GCC CTT TAG RRC AMA AAA TCC AGA MVT RGT YAT CTG | 82 | 3056 → 3091 |
| 31 | Y188 | Tag- CAG AAA TRG TYA TCT RTC AAT AYR TRG ATG AYT TRT A | 83 | 3076 → 3112 |
| 32 | 188L | Tag- CAG AAA TRG TYA TCT RTC AAT AYR TRG ATG AYT TRC T | 93 | 3076 → 3112 |
| 33 | G190 | Tag- ATA GTY ATC TRT CAA TAT RTG GAT GAC TTR TAT GTR GG | 97 | 3081 → 3118 |
| 34 | 190A | Tag- ATA GTY ATC TRT CAA TAT RTG GAT GAC TTR TAT GTR GC | 76 | 3081 → 3118 |
| 35 | V32 | Tag- CTC TYT TAG AYA CAG GAG CAG ATG AYA CAG | 14 | 2317 → 2346 |
| 36 | 37I | Tag- CTC TYT TAG AYA CAG GAG CAG ATG AYA CAA | 48 | 2317 → 2346 |
| 37 | I47 | Tag- TGC CAG GRA RAT GGA AAC CAA RAA TRA | 30 | 2365 → 2391 |
| 38 | 47V | Tag- GCC AGG RAR ATG GAA ACC AAR AAT RGT | 43 | 2366 → 2392 |
| 39 | 47A | Tag- GCC AGG RAR ATG GAA ACC AAR AAT RGC | 78 | 2366 → 2392 |
| 40 | L76 | Tag- AAA TTT GTG GRA AAA ARG CTR TAG GTA CAG TRT | 28 | 2446 → 2478 |
| 41 | 76V | Tag- AAA TTT GTG GRA AAA ARG CTR TAG GTA CAG TRG | 70 | 2446 → 2478 |
| 42 | I84 | Tag- AGT ATT ART RGG RCC TAC ACC TGT CAA YA | 35 | 2474 → 2502 |
| 43 | 84V | Tag- AGT ATT ART RGG RCC TAC ACC TGT CAA YG | 77 | 2474 → 2502 |
| 44 | L90 | Tag- CCT ACA CCT GTC AAC ATA ATT GGR AGR AAY HTR T | 95 | 2487 → 2520 |
| 45 | 90M | Tag- CCT ACA CCT GTC AAC ATA ATT GGR AGR AAY HTR A | 57 | 2487 → 2520 |

Tag, allele specific sequence as described herein
H = A, C, or T/U; K = G or T/U; M = A or C; N = A, C, G, or T/U; R = G or A; V = A, C, or G; W = A or T/U; Y = C or T/U
Locations indicated with respect to SEQ ID NO: 46 (HXB2: Genbank accession number K03455)

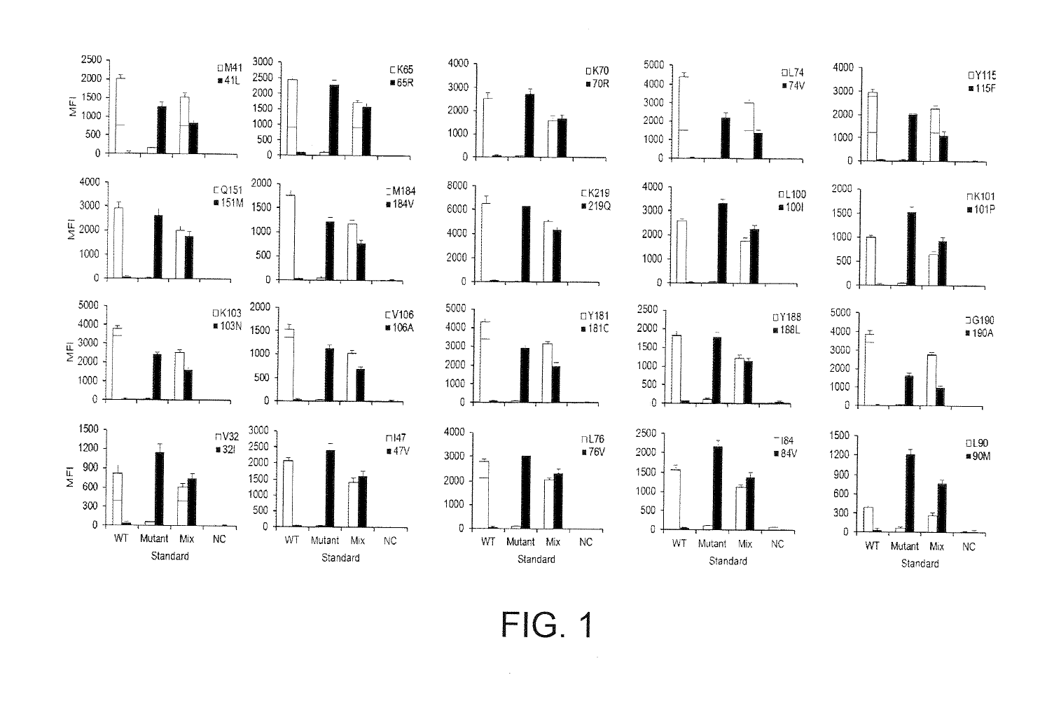

For initial development of the assay, two template DNAs were used. A first plasmid containing a partial HIV-1 pot gene fragment having the majority of RT, PI, and INT (integrase) resistant mutations was synthesized for use as an exemplary mutant template. The original plasmid DNA containing the wild type partial HIV-1 pol gene fragment was used as an exemplary wild type template. After PCR amplification of each template individually, wild type template, mutant template, a 1:1 mixture of the wild type and mutant templates, and a blank control were analyzed using all 45 ASPE primers in one reaction. FIG. 1 shows the results from five independent assays. Each of the wild type and mutant alleles (M41L, K65R, K70R, L74V, Y115F, Q151M, M184V, K219E, L100I, K101E, K103N, V106A, Y181C, Y188L, G190A, V32I, I47V, L76V, I84V and L90M) were correctly detected. These results demonstrate that as man as 20 alleles (both wild type and mutant) can be reliably and reproducibly detected by MASHMA.

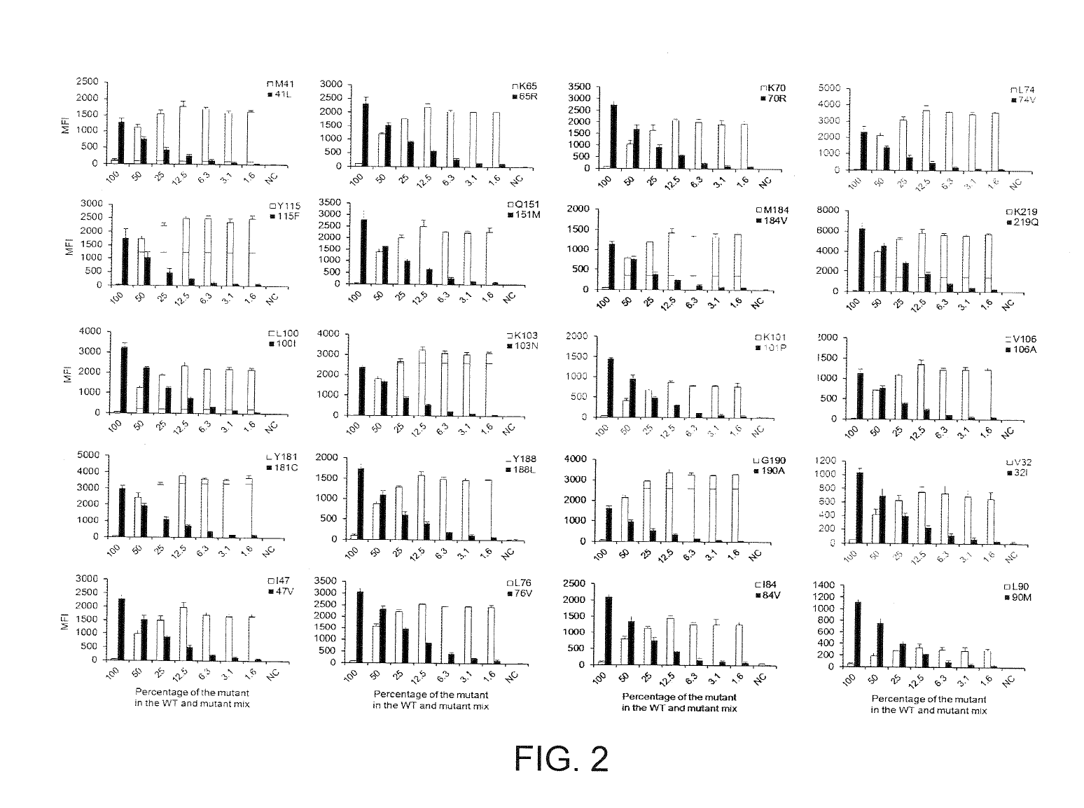

To determine the sensitivity of MASHMA, serial dilutions (1:2 ratio) of the mutant in the wild type template background were assayed using all 45 ASPE primers in one reaction. FIG. 2 shows the results from three independent assays. A significant concentration-effect relationship was observed. These results demonstrate that the detection limit of mutant alleles in the population is about 7%, which is more sensitive when compared to the sequencing method (˜20% detection limit).

### Example 2

### Rapid Detection of HIV-1 Drug Resistant Mutations in Clinical Samples

The assay developed as described in Example 1 was used to assess HIV-1 drug resistant mutations in clinical samples. Plasma samples were collected from 14 treated patients who were infected with subtype C viruses. Viral RNA was extracted from 200 μL of plasma samples using the NUCLISENS® EASYMAG® (Biomerieux) automated extraction system following the manufacturer's instructions. Reverse transcription and nested RT-PCR were performed essentially as described in Zhou et al., PLoS One, 2011, 6:e28184. The PCR amplified products were visualized on agarose gel (1.0%) and cleaned up using USB® EXOSAP-IT® kit (Affymetrix) (37° C. for 15 minutes and then 80° C. for additional 15 minutes). 5 μl of USB® EXOSAP-IT®-treated PCR products were used for Multiplex Allele Specific Primer Extension (mASPE) in 20 μl of solution containing 1×ASPE Buffer (2 mM Tris-HCl, pH 8.4; 5 mM KCl), 1.25 mM MgCl₂, primer mix (9.5×10¹¹ copies of each TAG-ASPE primer), 1.5 U Tsp DNA polymerase, 10 μM dATP, dTTP, dGTP, 10 μM biotin-dCTP and H₂O up to 20 μL. The mASPE reaction conditions used were: one cycle of 96° C. for 2 minutes; and 30 cycles of 94° C. for 30 seconds, 55° C. for 1 minute and 74° C. for 2 minutes. The appropriate MAGPLEX®-TAG microsphere sets (Luminex Corporation) were re-suspended by vortex for 20 seconds. 10 μl of mASPE products were mixed with MAGPLEX®-TAG mix (2.2×10³/μL of each microspheres set) in Tm Buffer (0.2 M Tris-HCl, pH 8.0, 0.4 M NaCl, 0.16% TRITON-X® 100) in 50 μl volume. The reaction was first denatured at 96° C. for 90 seconds and then annealed at 37° C. for 30 minutes.

The MAGPLEX®-TAG microspheres were pelleted by placing the plate on a magnetic separator for 60 seconds. After the supernatant was removed, microspheres were re-suspended in 100 μL of 1×Tm Hybridization Buffer (0.1 M Tris-HCl, pH 8.0, 0.2 M NaCl, 0.08% TRITON-X® 100) containing 4 μg/mL streptavidin-R-phycoerythrin and incubated at 37° C. for 15 minutes. 50 μL of the final reaction was analyzed on a LUMINEX® analyzer to determine the median fluorescence intensity (MFI) value for a specific bead region (or each allele at each mutation site). The MFI values for samples are corrected by subtracting the values of the bead control, and the blank control was used to ensure that contamination and unspecific signals were not introduced during the multiplex PCR and ASPE process. The MFI value for one allele was expected to be ≧180 and ≧3 times of the MFI of the no-target control for the same allele. If these criteria are not met, the analyzed allele was defined as signal too low to determine.

The results obtained by MASHMA were directly compared to those obtained by widely used population sequencing method as described in Zhou et al., PLoS One, 2011, 6:e28184. In half of the samples (7), complete concordant results for all 20 alleles were observed between the two methods. Only a few discordant results were observed between two methods: one allele difference in 5 samples, and two allele differences in 2 samples (See Tables 4A-4C). Overall, among 272 alleles that have measurable results for both methods, 269 were concordant (98.89%) (See Table 4D). Only 3 alleles were discordant. Two were a mixture of the wild type and mutant at NNRTI resistant mutation alleles by MASHMA but only the wild type by sequencing, while the other one a mixture of the wild type and mutant at an NRTI resistant mutation site by sequencing but only the wild type by MASHMA (See Tables 4D and 4E). MASHMA and sequencing methods showed complete concordant results for the majority of alleles tested (M41L, K65R, K70R, L74V, Y115F, I84V, L90M, K219Q/E, K101P/E, K103N/R, V106A/M, Y188L, V32I, and I47AV). Only at 6 alleles (Q151M, M184V, L100, Y181C, G190A and L76V), a few discordant results were observed (See Table 4F). Overall, of 280 alleles analyzed in 14 samples (20 alleles in each sample), 269 alleles (96.07%) were completely concordant between the MASHMA and sequencing methods. Among discordant results at 11 alleles, the majority of them (8) were due to the weak signals for which the identities of the base at these alleles could not be determined (See Tables 4A-4F). The data demonstrated that results from the MASHMA analysis is in excellent agreement with those from the population sequencing method, and that MASHMA offers benefits including high throughput, ease of performance and data interpretation, low cost, and the ability to determine multiple mutations in a large number of samples.

TABLE 4A
Detection of NRTI Resistant Mutations
by MASHMA and Population Sequencing

| Sample | NRTI Resistant Mutation | | | | | | | |
|---|---|---|---|---|---|---|---|---|
| ID | 41 | 65 | 70 | 74 | 115 | 151 | 184 | 219 |
| 1 | wt | wt | wt | wt | wt | wt | wt | wt |
| 2 | wt | wt | wt | wt | wt | wt | M184V | wt |
| 3 | wt | wt | wt | wt | wt | wt | M184V | wt |
| 4 | wt | wt | wt | wt | wt | wt | M184V | wt |
| 5 | wt | wt | wt | wt | wt | wt | wt | wt |
| 6 | wt | wt | K70R | wt | wt | wt | M184V | K219E |
| 7 | wt | wt | wt | wt | wt | wt | M184V | wt |
| 8 | wt | wt | wt | wt | wt | ND | wt | wt |
| 9 | wt | wt | wt | wt | wt | ND | M184V | wt |
| 10 | wt | wt | wt | wt | wt | wt | M184V | wt |
| 11 | wt | wt | wt | wt | wt | wt | wt | wt |
| 12 | wt | wt | wt | wt | wt | ND | M184V | wt |
| 13 | wt | K65R | wt | wt | wt | wt | M184+ | wt |
| 14 | wt | wt | wt | wt | wt | wt | ND | wt |

wt, wild type
ND, signal too low for allele determination
+both wild type and M184V were detected by sequencing; same results were obtained by MASHMA and sequencing methods for all other alleles

TABLE 4B
Detection of NNRTI Resistant Mutations
by MASHMA and Population Sequencing

| Sample | NNRTI Resistant Mutation | | | | | | |
|---|---|---|---|---|---|---|---|
| ID | 100 | 101 | 103 | 106 | 181 | 188 | 190 |
| 1 | wt | wt | wt | wt | Y181YC | wt | wt |
| 2 | wt | wt | K103N | wt | wt | wt | wt |
| 3 | wt | wt | K103KN | wt | Y181C | wt | wt |
| 4 | wt | wt | wt | wt | Y181C | wt | wt |
| 5 | wt | wt | wt | wt | wt | Y188L | wt |
| 6 | wt | wt | wt | wt | Y181C | wt | wt |
| 7 | wt | wt | K103N | wt | wt | wt | wt |
| 8 | wt | wt | wt | V106A | wt | wt | wt |
| 9 | wt | wt | K103N | wt | Y181YC* | wt | wt |
| 10 | ND | K101E | wt | wt | wt | wt | G190GA* |
| 11 | wt | wt | K103N | wt | ND | wt | wt |
| 12 | wt | wt | K103N | wt | Y181C | wt | wt |
| 13 | wt | wt | wt | wt | Y181C | wt | wt |
| 14 | wt | wt | K103N | wt | ND | wt | wt |

wt, wild type
ND, signal too low for allele determination
*mix of wild type and mutant bases were detected by MASHMA, while sequencing result only detected the wild type (Y181) or the mutant (G190A); same results were obtained by MASHMA and sequencing methods for all other alleles

TABLE 4C
Detection of PI Resistant Mutations
by MASHMA and Population Sequencing

| Sample | PI | | | | |
|---|---|---|---|---|---|
| ID | 32 | 47 | 76 | 84 | 90 |
| 1 | wt | wt | wt | wt | wt |
| 2 | wt | wt | wt | wt | wt |
| 3 | wt | wt | wt | wt | wt |
| 4 | wt | wt | wt | wt | wt |
| 5 | wt | wt | wt | wt | wt |
| 6 | wt | wt | wt | wt | wt |
| 7 | wt | wt | wt | wt | wt |
| 8 | wt | wt | wt | wt | wt |
| 9 | wt | wt | wt | wt | wt |
| 10 | wt | wt | wt | wt | wt |
| 11 | wt | wt | wt | wt | wt |
| 12 | wt | wt | wt | wt | wt |
| 13 | wt | wt | ND | wt | wt |
| 14 | wt | wt | wt | wt | wt |

wt, wild type
ND, signal too low for allele determination; same results were obtained by MASHMA and sequencing methods for all other alleles

TABLE 4D
Summary of Drug Resistant Mutations Detected
by MASHMA and Population Sequencing

Sequencing

| | | wt | mut | mix |
|---|---|---|---|---|
| MASHMA | wt | 241 | | 1 |
| | mut | | 26 | |
| | mix | 2 | | 2 |
| | ND | 8 | | |

wt, wild type
mut, mutant
mix, mixture of wild type and mutant
ND, signal too low for allele determination

TABLE 4E
Summary of NRTI, NNRTI, and PI Resistant Mutations
Detected by MASHMA and Population Sequencing

| | Sequencing | | | | | | | | | |
|---|---|---|---|---|---|---|---|---|---|---|
| | | NRTI | | | NNRTI | | | PI | | |
| | | wt | mut | mix | wt | mut | mix | wt | mut | mix |
| MASHMA | wt | 96 | | 1 | 76 | | | 69 | | |
| | mut | | 11 | | | 15 | | | | |
| | mix | | | | 2 | | 2 | | | |
| | ND | 4 | | | 3 | | | 1 | | |

wt, wild type
mut, mutant
mix, mixture of wild type and mutant
ND, signal too low for allele determination

TABLE 4F
Summary of Individual Drug Resistant Mutations
Detected by MASHMA and Population Sequencing

| | Sequencing | | | | | | | | | | | | | | | |
|---|---|---|---|---|---|---|---|---|---|---|---|---|---|---|---|---|
| | | wt | mut | mix | wt | mut | mix | wt | mut | mix | wt | mut | mix | wt | mut | mix |
| | | M41L* | | | K65R* | | | K70R* | | | L74V* | | | Y115F* | | |
| MASHMA | wt | 14 | | | 13 | | | 13 | | | 14 | | | 14 | | |
| | mut | | | | | 1 | | | 1 | | | | | | | |
| | mix | | | | | | | | | | | | | | | |
| | ND | | | | | | | | | | | | | | | |
| | | Q151M | | | M184V | | | I84V* | | | L90M* | | | K219Q/E* | | |
| | wt | 11 | | | 4 | | 1 | 14 | | | 14 | | | 13 | | |
| | mut | | | | | 8 | | | | | | | | | 1 | |
| | mix | | | | | | | | | | | | | | | |
| | ND | 3 | | | 1 | | | | | | | | | | | |
| | | L100I | | | K101P/E* | | | K103N/R* | | | V106A/M* | | | Y181C | | |
| | wt | 13 | | | 13 | | | 7 | | | 13 | | | 5 | | |
| | mut | | | | | 1 | | | 6 | | | 1 | | | 5 | |
| | mix | | | | | | | | | 1 | | | | 1 | | 1 |
| | ND | 1 | | | | | | | | | | | | 2 | | |
| | | Y188L* | | | G190A | | | V32I* | | | I47A/V | | | L76V | | |
| | wt | 13 | | | 13 | | | 14 | | | 14 | | | 13 | | |
| | mut | | 1 | | | | | | | | | | | | | |
| | mix | | | | 1 | | | | | | | | | | | |
| | ND | | | | | | | | | | | | | 1 | | |

wt, wild type
mut, mutant
mix, mixture of wild type and mutant
ND, signal too low for allele determination
*concordant results between MASHMA and sequencing methods

1. A method of genotyping two or more polymorphic bases at two or more loci of a target nucleic acid, the method comprising the steps of:
(a) obtaining a target nucleic acid;
(b) preparing a reaction mixture comprising the target nucleic acid and a plurality of allele specific primer extension primers that specifically hybridize to the two or more loci of the target nucleic acid, in conditions sufficient for hybridization, primer extension, and labeling with a reporter molecule;
wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence;
(c) annealing the primer extension products of (b) to a plurality of beads, wherein each bead comprises (i) a second unique allele specific sequence specifically hybridizes to the first unique allele specific sequence, and (ii) a unique detectable label;
(d) detecting the reporter molecule; and
(e) detecting the unique detectable label.

2. The method of claim 1, wherein the target nucleic acid is associated with drug resistance.

3.-6. (canceled)

7. The method of claim 2, wherein the target nucleic acid is a pathogen nucleic acid.

8.-12. (canceled)

13. The method of claim 1, wherein the target nucleic acid is obtained from a human subject.

14. The method of claim 13, wherein the human subject has received antiretroviral therapy.

15.-16. (canceled)

17. The method of claim 13, wherein the human subject is an HIV-infected patient.

18. (canceled)

19. The method of claim 1, wherein the plurality of all allele specific primer extension primers comprises two or more primers that specifically hybridize to polymorphic bases of a pathogen target nucleic acid, which polymorphic bases are at two or more loci corresponding to drug resistant mutations of the target nucleic acid.

20. The method of claim 19, wherein the plurality of allele specific primer extension primers comprises primers that specifically hybridize to a polymorphic base selected from any one of V32I, M41L, I47A/V, K65R, D67N, K70R, L74V, L76V, I84V, L90M, L100I, K101P, K103N, V106A/M, Y115F, Q151M, Y181C, M184V, Y188L, G190A, L210W, T215F/Y, and K219Q/E of an HIV-1 pol gene.

21.-23. (canceled)

24. The method of claim 1, wherein the plurality of allele specific primer extension primers that specifically hybridize to the two or more loci comprises at least one allele specific primer extension primer set, wherein a primer set comprises at least a first primer that hybridizes to a wild type allele comprising a first polymorphic base at a locus and a second primer that hybridizes to a mutant allele comprising a second polymorphic base at the same locus.

25.-26. (canceled)

27. The method of claim 1, wherein the reporter molecule comprises a biotinylated deoxynucleotide.

28.-29. (canceled)

30. The method of claim 1, wherein detecting the unique detectable label comprises laser-based fluorescent analysis.

31. A composition comprising a plurality of allele specific primer extension primers that specifically hybridize to two or more loci of a target nucleic acid, wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence.

32. The composition of claim 31, wherein the target nucleic acid is associated with drug resistance.

33.-42. (canceled)

43. The composition of claim 31, wherein the plurality of allele specific primer extension primers comprises two or more primers that specifically hybridize to polymorphic bases of a pathogen target nucleic acid, which polymorphic bases are at two or more loci corresponding to drug resistant mutations of the target nucleic acid.

44. The composition of claim 43, wherein the plurality of allele specific primer extension primers comprises primers that specifically hybridize to a polymorphic base selected from any one of V32I, M41L, I47A/V, K65R, D67N, K70R, L74V, L76V, I84V, L90M, L100I, K101P, K103N, V106A/M, Y115F, Q151M, Y181C, M184V, Y188L, G190A, L210W, T215F/Y, and K219Q/E of an HIV-1 pol gene.

45.-47. (canceled)

48. The composition of claim 31, wherein the plurality of allele specific primer extension primers that specifically hybridize to the two or more loci comprises at least one allele specific primer extension primer set, wherein a primer set comprises at least a first primer that hybridizes to a wild type allele comprising a first polymorphic base at a locus and a second primer that hybridizes to a mutant allele comprising a second polymorphic base at the same locus.

49.-50. (canceled)

51. A kit comprising:
(a) a plurality of allele specific primer extension primers that specifically hybridize to two or more loci of a target nucleic acid, wherein each primer (i) specifically hybridizes to a wild type allele or to a mutant allele comprising a polymorphic base, (ii) comprises at its 3′ end, a nucleotide that is complementary to the polymorphic base, and (iii) comprises at or near its 5′ end, a first unique allele specific sequence; and
(b) a plurality of beads, wherein each bead comprises (i) a second unique allele specific sequence that is reverse complementary to the first unique allele specific sequence, and (ii) a unique detectable label.

52. The kit of claim 51, wherein the target nucleic acid is associated with drug resistance.

53.-62. (canceled)

63. The kit of claim 51, wherein the plurality of allele specific primer extension primers comprises two or more primers that specifically hybridize to polymorphic bases of a pathogen target nucleic acid, which polymorphic bases are at loci corresponding to drug resistant mutations of the target nucleic acid.

64.-67. (canceled)

68. The kit of claim 51, wherein the plurality of allele specific primer extension primers that specifically hybridize to the two or more loci comprises at least one allele specific primer extension primer set, wherein a primer set comprises at least a first primer that hybridizes to a wild type allele comprising a first polymorphic base at a locus and a second primer that hybridizes to a mutant allele comprising a second polymorphic base at the same locus.

69.-70. (canceled)

71. The kit of claim 51, further comprising a reporter molecule, wherein the reporter molecule for labeling of allele specific primer extension reaction products.

72.-75. (canceled)


SEQUENCE LISTING (49 sequences)

SEQ ID NO: 1 - DNA, 39 bp, Artificial Sequence
  Description of Artificial Sequence Synthetic ASPE primer
aagaraaaataaaagcattaayagmaatttgtgawgara

SEQ ID NO: 2 - DNA, 39 bp, Artificial Sequence
  Description of Artificial Sequence Synthetic ASPE primer
aagaraaaataaaagcattaayagmaatttgtgawgarc

SEQ ID NO: 3 - DNA, 36 bp, Artificial Sequence
  Description of Artificial Sequence Synthetic ASPE primer
aaatccatataacactccartatttgcyataaaraa

SEQ ID NO: 4 - DNA, 36 bp, Artificial Sequence
  Description of Artificial Sequence Synthetic ASPE primer
aaatccatataacactccartatttgcyataaarag

SEQ ID NO: 5 - DNA, 35 bp, Artificial Sequence
  Description of Artificial Sequence Synthetic ASPE primer
ccagtatttgccataaagargaargayagtactaa

SEQ ID NO: 6 - DNA, 35 bp, Artificial Sequence
  Description of Artificial Sequence Synthetic ASPE primer
ccagtatttgccataaagargaargayagtactag

SEQ ID NO: 7 - DNA, 35 bp, Artificial Sequence
  Description of Artificial Sequence Synthetic ASPE primer
cataaaaaagaargacagtacharrtggagaaaat

SEQ ID NO: 8 - DNA, 35 bp, Artificial Sequence
  Description of Artificial Sequence Synthetic ASPE primer
cataaaaaagaargacagtacharrtggagaaaag

SEQ ID NO: 9 - DNA, 24 bp, Artificial Sequence
  Description of Artificial Sequence Synthetic ASPE primer
agtrctrgaygtgggrgatgcata

SEQ ID NO: 10 - DNA, 24 bp, Artificial Sequence
  Description of Artificial Sequence Synthetic ASPE primer
agtrctrgaygtgggrgatgcatt

SEQ ID NO: 11 - DNA, 30 bp, Artificial Sequence
  Description of Artificial Sequence Synthetic ASPE primer
ggattagrtatcaatataatgtrytnccac

SEQ ID NO: 12 - DNA, 30 bp, Artificial Sequence
  Description of Artificial Sequence Synthetic ASPE primer
ggattagrtatcaatataatgtrytnccaa

SEQ ID NO: 13 - DNA, 37 bp, Artificial Sequence
  Description of Artificial Sequence Synthetic ASPE primer
agrgcaaaaaatccagamrtrgtyatctrycaataya

SEQ ID NO: 14 - DNA, 37 bp, Artificial Sequence
  Description of Artificial Sequence Synthetic ASPE primer
agrgcaaaaaatccagamrtrgtyatctrycaatayg

SEQ ID NO: 15 - DNA, 25 bp, Artificial Sequence
  Description of Artificial Sequence Synthetic ASPE primer
aartggggrtttacyacaccagaca

SEQ ID NO: 16 - DNA, 25 bp, Artificial Sequence
  Description of Artificial Sequence Synthetic ASPE primer
aartggggrtttacyacaccagacc

SEQ ID NO: 17 - DNA, 25 bp, Artificial Sequence
  Description of Artificial Sequence Synthetic ASPE primer
aartggggrtttacyacaccagakg

SEQ ID NO: 18 - DNA, 28 bp, Artificial Sequence
  Description of Artificial Sequence Synthetic ASPE primer
caattaggrataccacacccakcaggrt

SEQ ID NO: 19 - DNA, 28 bp, Artificial Sequence
  Description of Artificial Sequence Synthetic ASPE primer
caattaggrataccacacccakcaggra

SEQ ID NO: 20 - DNA, 30 bp, Artificial Sequence
  Description of Artificial Sequence Synthetic ASPE primer
aattaggrataccacacccakcaggrwtra

SEQ ID NO: 21 - DNA, 30 bp, Artificial Sequence
  Description of Artificial Sequence Synthetic ASPE primer
aattaggrataccacacccakcaggrwtrc

SEQ ID NO: 22 - DNA, 30 bp, Artificial Sequence
  Description of Artificial Sequence Synthetic ASPE primer
aattaggrataccacacccakcaggrwtrg

SEQ ID NO: 23 - DNA, 32 bp, Artificial Sequence
  Description of Artificial Sequence Synthetic ASPE primer
gaataccacacccakcagggttraaraagaaa

SEQ ID NO: 24 - DNA, 32 bp, Artificial Sequence
  Description of Artificial Sequence Synthetic ASPE primer
gaataccacacccakcagggttraaraagaay

SEQ ID NO: 25 - DNA, 32 bp, Artificial Sequence
  Description of Artificial Sequence Synthetic ASPE primer
gaataccacacccakcagggttraaraagaga

SEQ ID NO: 26 - DNA, 33 bp, Artificial Sequence
  Description of Artificial Sequence Synthetic ASPE primer
acacccakcagggttaaaraagaahaartcwgt

SEQ ID NO: 27 - DNA, 33 bp, Artificial Sequence
  Description of Artificial Sequence Synthetic ASPE primer
acacccakcagggttaaaraagaahaartcwgc

SEQ ID NO: 28 - DNA, 33 bp, Artificial Sequence
  Description of Artificial Sequence Synthetic ASPE primer
cacacccakcagggttaaaraagaahaartcwa

SEQ ID NO: 29 - DNA, 36 bp, Artificial Sequence
  Description of Artificial Sequence Synthetic ASPE primer
gccctttagrrcamaaaatccagamvtrgtyatcta

SEQ ID NO: 30 - DNA, 36 bp, Artificial Sequence
  Description of Artificial Sequence Synthetic ASPE primer
gccctttagrrcamaaaatccagamvtrgtyatctg

SEQ ID NO: 31 - DNA, 37 bp, Artificial Sequence
  Description of Artificial Sequence Synthetic ASPE primer
cagaaatrgtyatctrtcaatayrtrgatgayttrta

SEQ ID NO: 32 - DNA, 37 bp, Artificial Sequence
  Description of Artificial Sequence Synthetic ASPE primer
cagaaatrgtyatctrtcaatayrtrgatgayttrct

SEQ ID NO: 33 - DNA, 38 bp, Artificial Sequence
  Description of Artificial Sequence Synthetic ASPE primer
atagtyatctrtcaatatrtggatgacttrtatgtrgg

SEQ ID NO: 34 - DNA, 38 bp, Artificial Sequence
  Description of Artificial Sequence Synthetic ASPE primer
atagtyatctrtcaatatrtggatgacttrtatgtrgc

SEQ ID NO: 35 - DNA, 30 bp, Artificial Sequence
  Description of Artificial Sequence Synthetic ASPE primer
ctctyttagayacaggagcagatgayacag

SEQ ID NO: 36 - DNA, 30 bp, Artificial Sequence
  Description of Artificial Sequence Synthetic ASPE primer
ctctyttagayacaggagcagatgayacaa

SEQ ID NO: 37 - DNA, 27 bp, Artificial Sequence
  Description of Artificial Sequence Synthetic ASPE primer
tgccaggraratggaaaccaaraatra

SEQ ID NO: 38 - DNA, 27 bp, Artificial Sequence
  Description of Artificial Sequence Synthetic ASPE primer
gccaggraratggaaaccaaraatrgt

SEQ ID NO: 39 - DNA, 27 bp, Artificial Sequence
  Description of Artificial Sequence Synthetic ASPE primer
gccaggraratggaaaccaaraatrgc

SEQ ID NO: 40 - DNA, 33 bp, Artificial Sequence
  Description of Artificial Sequence Synthetic ASPE primer
aaatttgtggraaaaargctrtaggtacagtrt

SEQ ID NO: 41 - DNA, 33 bp, Artificial Sequence
  Description of Artificial Sequence Synthetic ASPE primer
aaatttgtggraaaaargctrtaggtacagtrg

SEQ ID NO: 42 - DNA, 29 bp, Artificial Sequence
  Description of Artificial Sequence Synthetic ASPE primer
agtattartrggrcctacacctgtcaaya

SEQ ID NO: 43 - DNA, 29 bp, Artificial Sequence
  Description of Artificial Sequence Synthetic ASPE primer
agtattartrggrcctacacctgtcaayg

SEQ ID NO: 44 - DNA, 34 bp, Artificial Sequence
  Description of Artificial Sequence Synthetic ASPE primer
cctacacctgtcaacataattggragraayhtrt

SEQ ID NO: 45 - DNA, 34 bp, Artificial Sequence
  Description of Artificial Sequence Synthetic ASPE primer
cctacacctgtcaacataattggragraayhtra

SEQ ID NO: 46 - DNA, 9719 bp, Human immunodeficiency virus type 1
  CDS (790)..(2289): gag polyprotein
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaccctgtgagcctgcatggaatggatgacccggagagagaagtgttagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 47 - protein, 500 aa, Human immunodeficiency virus type 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 48 - protein, 912 aa, Human immunodeficiency virus type 1
MSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 49 - protein, 856 aa, Human immunodeficiency virus type 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL